(19) United States
(12) Patent Application Publication
(10) Pub. No.: US 2020/0045985 A1
(43) Pub. Date: Feb. 13, 2020

(54) CHOLINE ESTER-CONTAINING COMPOSITION FOR ORAL INGESTION

(71) Applicant: Shinshu University, Matsumoto-shi, Nagano (JP)

(72) Inventor: Kozo Nakamura, Kamiina-gun, Nagano (JP)

(73) Assignee: Shinshu University, Matsumoto-shi, Nagano (JP)

(21) Appl. No.: 16/341,748

(22) PCT Filed: Oct. 16, 2017
(86) PCT No.: PCT/JP2017/037389

§ 371 Date: Apr. 12, 2019

(30) Foreign Application Priority Data
Oct. 14, 2016 (JP) 2016-202909

(51) Int. Cl. A23C 3/02; A23C 9/123

(52) U.S. Cl. CPC A23C 3/02; A23C 9/123

(57) ABSTRACT

The present invention addresses the problem of solving problems of the conventional techniques and producing, by an economically advantageous means, fermented milk having hardness to such an extent that the texture of the fermented milk can be kept during distribution. The present invention relates to a method for producing fermented milk, comprising homogenizing a raw material mix under a high pressure to reduce the average particle diameter of a fat and then fermenting the homogenized product. More specifically, the present invention relates to: a method for producing fermented milk using a material that is sterilized at an ultra-high temperature, said method comprising homogenizing a fat in a raw material mix, which contains a material sterilized at an ultra-high temperature, under a high pressure and then fermenting the resultant product; and fermented milk produced by the method.

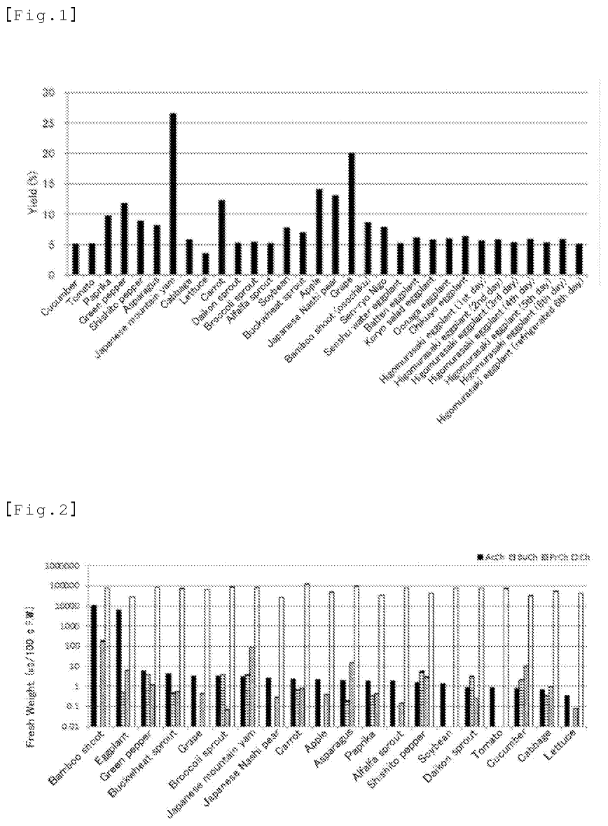

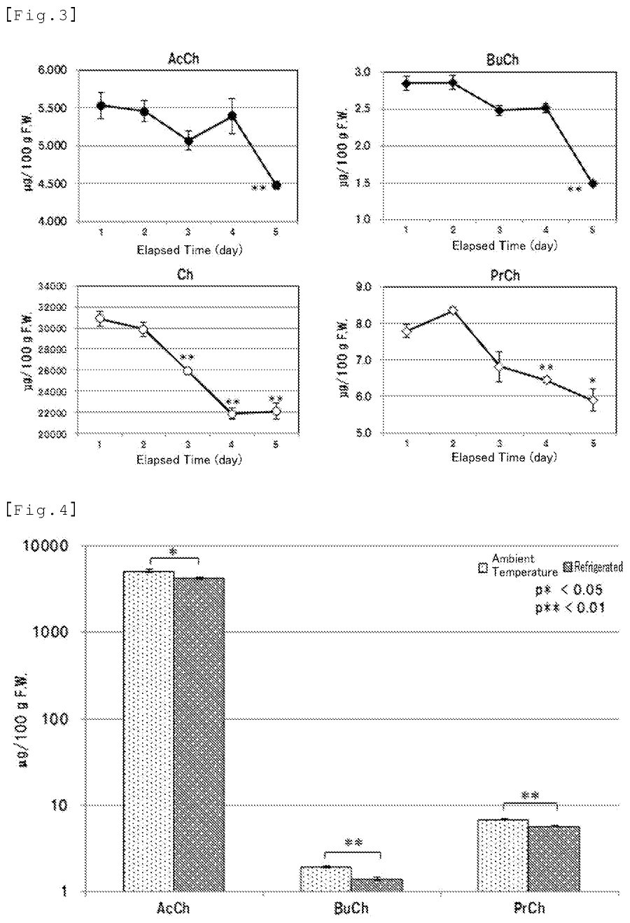

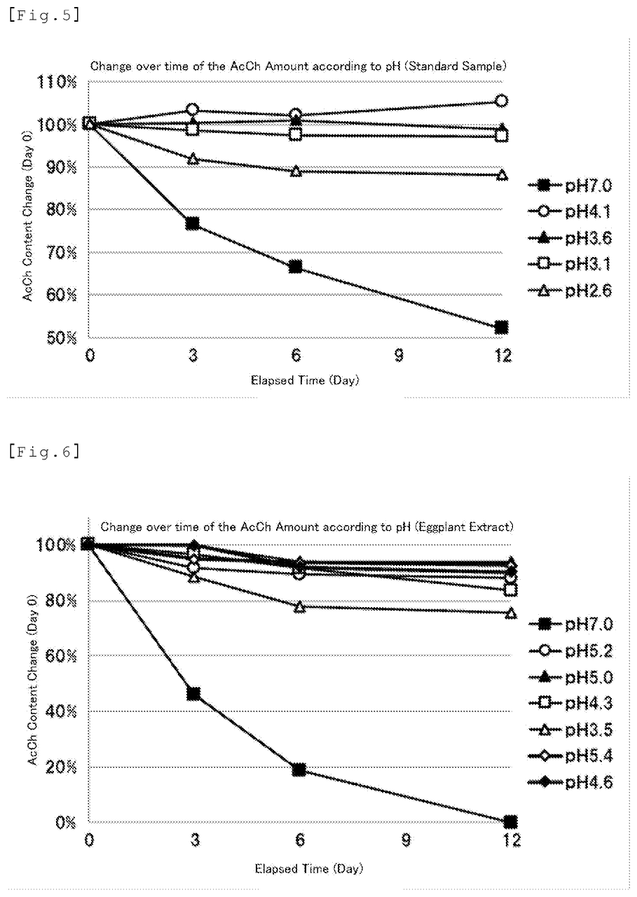

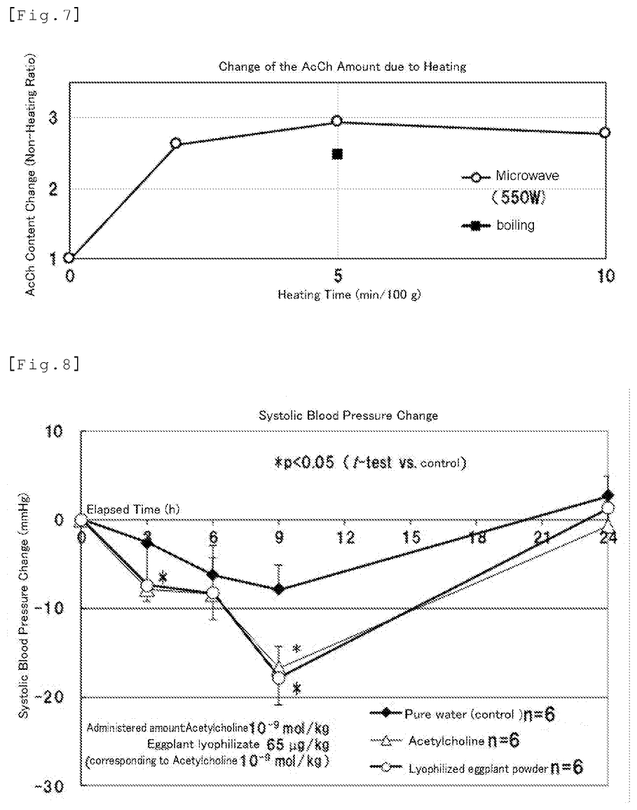

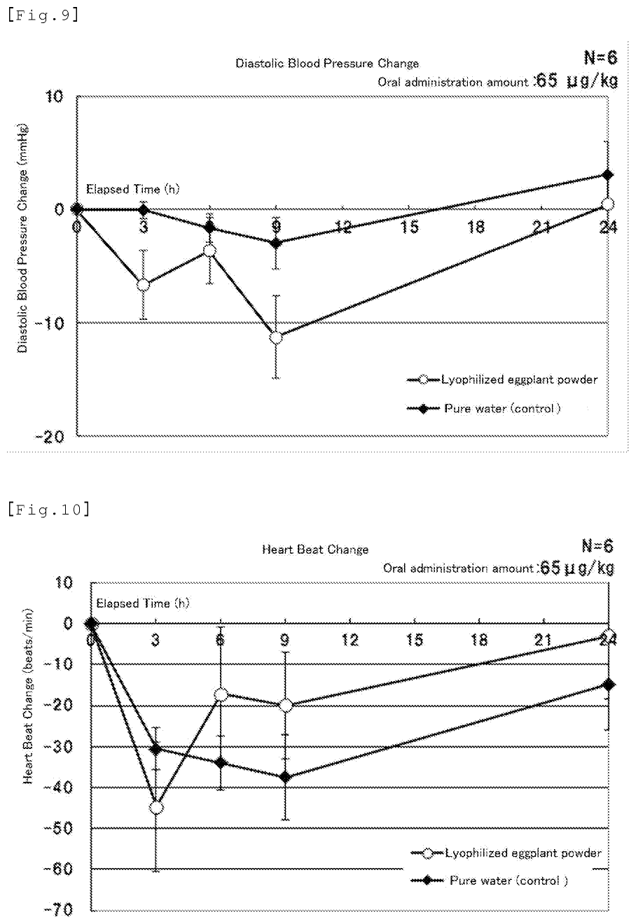

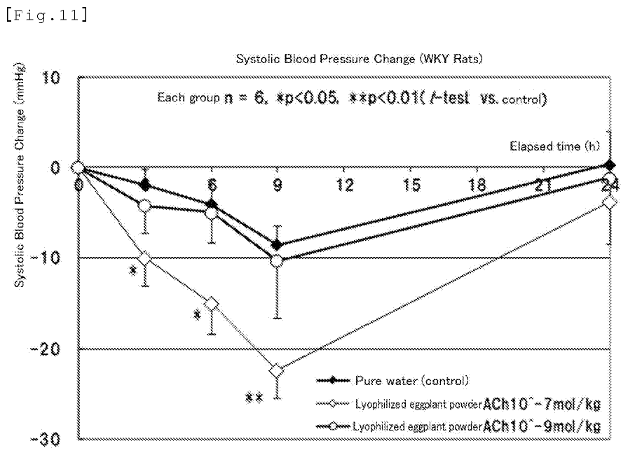

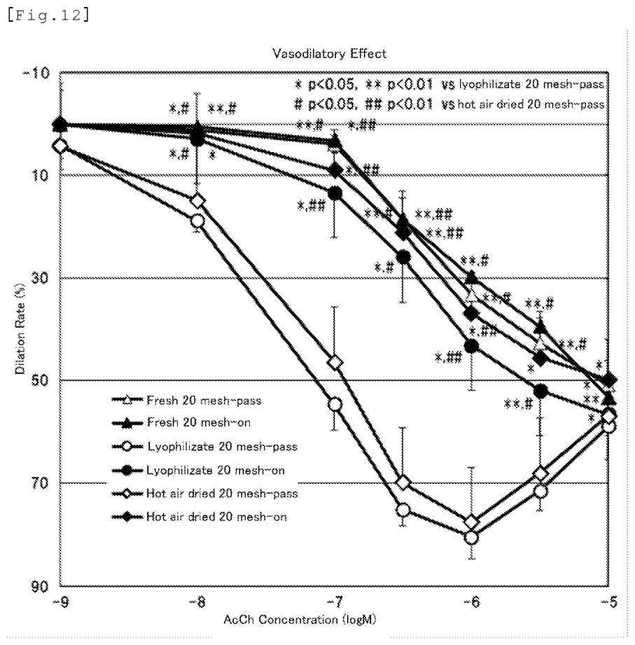

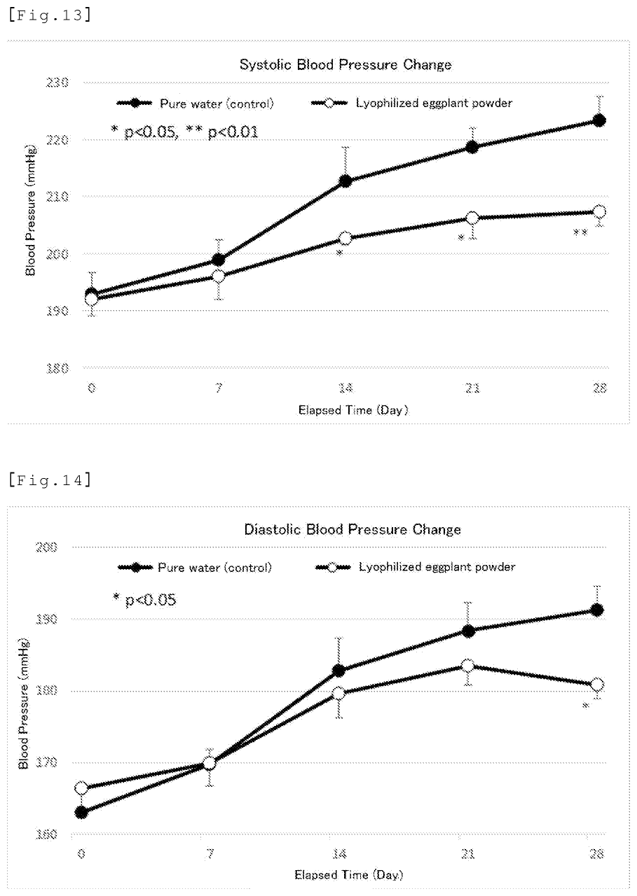

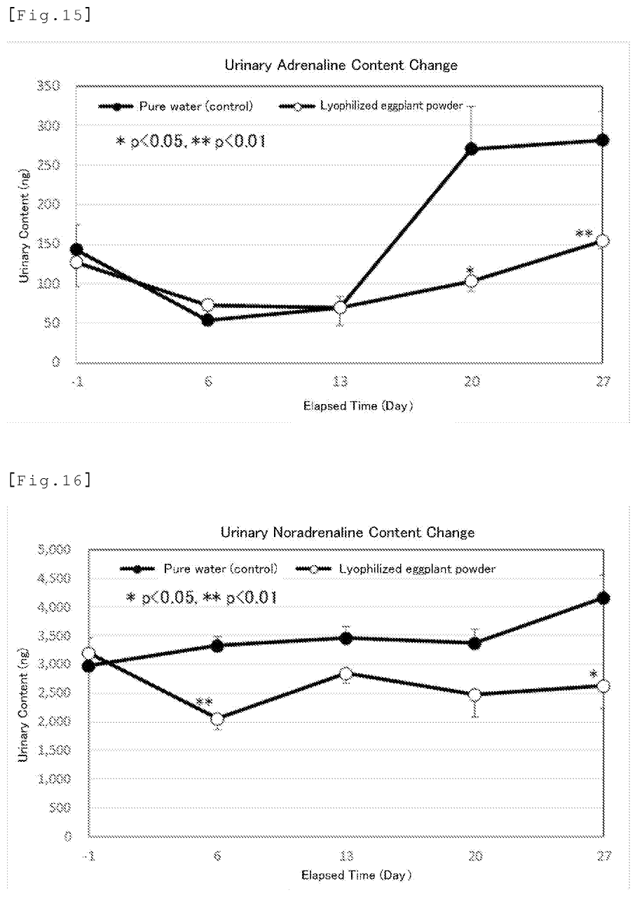

### TECHNICAL FIELD

The present invention relates to a method for producing fermented milk and fermented milk obtained by said production method.

### BACKGROUND ART

In dairy products such as milk, ultra-high temperature (UHT) treatment is generally carried out. Compared to high temperature/short time (HTST) treatment and low temperature/long time (LTLT) treatment, sterilization can be carried out in a short time, so there is an advantage that efficiency is good and thermal denaturation of milk protein is reduced. Meanwhile, a raw material mix for fermented milk is subjected to heat sterilization before fermentation, where such heat sterilization has been carried out by high temperature/short time (HTST) treatment. This is because yogurt having sufficient hardness as a commercial product could not be obtained since protein in the raw material mix was denatured when the raw material mix was exposed to a high temperature such as in ultra-high temperature (UHT) treatment (Patent Document 1). It is known that physical properties of yogurt curd are greatly influenced by thermal denaturation of whey protein (Non-Patent Documents 1 and 2).

Heretofore, as a method for producing fermented milk using high-temperature short-time sterilization treatment, the following method has been disclosed (Patent Documents 2 and 3): after reducing the dissolved oxygen concentration in a raw material mix, the raw material mix is sterilized at high temperature for a short time and then fermented at low temperature, thereby obtaining fermented milk having a hardness sufficient to maintain the texture during distribution; however, a facility for reducing dissolved oxygen concentration is required, and therefore it cannot be necessarily said that this method is economically advantageous to produce fermented milk.

### CITATION LIST

### Patent Documents

[Patent Document 1] WO 2008/068893
[Patent Document 2] JP No. 3644505
[Patent Document 3] JP No. 3666871

### Non-Patent Documents

[Non-Patent Document 1] A. E. LABROPOULOS et al., “WHEY PROTEIN DENATURATION OF UHT PROCESSED MILK AND ITS EFFECT ON RHEOLOGY OF YOGURT”, Journal of Texture Studies 12(3): 365-374, 1981.
[Non-Patent Document 2] Tomohiro Noguchi, “A new approach to coagulability of yogurt products—effect of degree of thermal denaturation on skimmed milk (powder)” Information on Livestock, Domestic Edition (202), pp. 26-29, August 2006 (in Japanese).

### SUMMARY OF INVENTION

### Problems to be Solved by Invention

Therefore, an object of the present invention is to solve the problems of the prior art, and to obtain fermented milk having a hardness sufficient to maintain the texture during distribution by a more economically advantageous means.

### Means of Solving Problems

The inventors of the present invention have conducted intensive studies to achieve the above-mentioned object, and found that the object can be achieved by homogenizing a raw material mix, particularly homogenizing at high pressure, thereby reducing the average particle diameter of the fat in the raw material mix.

Namely, the present invention relates to the following.

[1] A method for producing fermented milk using a material sterilized by ultra-high temperature treatment, comprising homogenizing the fat of a raw material mix containing the material sterilized by ultra-high temperature treatment, and conducting fermentation.
[2] The method according to the above [1], wherein the material sterilized by ultra-high temperature treatment is one or more selected from the group consisting of milk, concentrated milk, whole milk powder, skimmed milk, concentrated skimmed milk, skimmed milk powder, partially-skimmed milk, concentrated partially-skimmed milk, partially-skimmed milk powder, cream and butter.
[3] The method according to the above [1] or [2], wherein the temperature of the ultra-high temperature treatment is 120° C. to 150° C.
[4] The method according to any one of the above [1] to [3], wherein the fat in the raw material mix is homogenized such that its average particle diameter becomes 0.8 μm or less.
[5] The method according to any one of the above [1] to [4], further comprising heat sterilizing the raw material mix.
[6] The method according to any one of the above [1] to [5], further comprising reducing the dissolved oxygen concentration of the raw material mix.
[7] The method according to the above [6], comprising reducing the dissolved oxygen concentration of the raw material mix before heat sterilizing the raw material mix.
[8] The method according to the above [6] or [7], comprising reducing the dissolved oxygen concentration of the raw material mix before fermenting the raw material mix.
[9] The method according to any one of the above [1] to [8], wherein the fermentation of the raw material mix is carried out in a container of the product.
[10] Fermented milk produced by the method according to any one of the above [1] to [9].
[11] The fermented milk according to the above [10], wherein the fermented milk is a set type yogurt.
[12] The method according to the above [11], wherein the hardness is 26 g or more.

### Advantageous Effects of Invention

The present invention enables to increase the curd strength of fermented milk to such an extent that it can withstand vibrations during distribution, even when sufficient curd strength cannot be obtained due to insufficient denaturation (for example, low temperature sterilization treatment of a raw material mix) or due to excessive denaturation (for example, ultra-high temperature treatment of a raw material mix, or ultra-high temperature treatment of a part of materials of a raw material mix). Therefore, even when a raw material mix or a part of materials of a raw material mix has a thermal history of 63° C. to 150° C., it is possible to increase the curd strength of the fermented milk to such an extent that it can withstand vibrations during distribution.

Furthermore, according to the production method of the present invention, fermented milk with improved smoothness of mouth feel can be provided regardless of the thermal history of materials of a raw material mix or the thermal history of a raw material mix of the fermented milk.

In particular, the present invention can improve the deterioration of the curd strength, which occurs when fermented milk is produced by using a material sterilized by ultra-high temperature treatment, while imparting smooth mouth feel to the fermented milk, thereby providing fermented milk having curd strength to withstand the transport while having very smooth mouth feel. Furthermore, the present invention can enhance the physical properties of fermented milk without requiring additives for increasing the curd strength. In addition, the present invention also makes it possible to widely utilize raw materials sterilized by ultra-high temperature treatment in the production of fermented milk. That is, according to the conventional production method, when raw materials once heated and sterilized at an ultra-high temperature (for example, 120° C. or higher) are used for a raw material mix, the curd strength of the produced fermented milk is extremely low regardless of the subsequent thermal history; however, according to the present invention, regardless of the thermal history, raw materials sterilized by ultra-high temperature treatment can be used in the production of fermented milk.

Furthermore, when fermented after reducing the dissolved oxygen concentration, fermented milk having excellent denseness of the texture of the curd, mellowness of the aftertaste, creamy feeling of the flavor, richness feeling, milky feeling of the aftertaste, excellent smoothness and sense of filling, good flavor, and even better smoothness of mouth feel can be obtained.

### EMBODIMENTS FOR CARRYING OUT INVENTION

The present invention relates to a method for producing fermented milk using a material sterilized by ultra-high temperature treatment, the method comprising homogenizing the fat of a raw material mix containing the material sterilized by ultra-high temperature treatment, and conducting fermentation.

Fermented milk is the one which is prepared by fermenting milk, or a milk, etc. containing milk solids-not-fat at the same level as the milk, with lactic acid bacteria or yeast to make a paste, liquid or solid form, or a frozen matter thereof, and it can be roughly divided into two types. One is a pre-fermentation type, and the other is a post-fermentation type. In the former (pre-fermentation type), a predetermined amount of a starter (lactic acid bacteria, etc.) is added to a raw material mix, and the raw material mix is fermented using a tank prior to filling in a single-serving container for distribution until a predetermined level of lactic acid acidity or predetermined level of pH, etc. is reached, then after cooling, the obtained fermented milk is crushed etc. and mixed with flesh of fruit and a sweetener (sugar solution, etc.) as necessary, and is packed in a single-serving container (paper containers, plastic containers, glass containers, etc.). In the latter (post-fermentation type), a predetermined amount of a starter is added to a raw material mix, and the raw material mix is filled in a single-serving container for distribution, and this raw material mix is fermented using a fermentation chamber, etc. until a predetermined level of lactic acid acidity or predetermined level of pH, etc. is reached, then it is solidified to a purine form and cooled. Pre-fermentation is often used for the production of soft-type yogurt with flesh of fruit and drink type yogurt with sweetener. Whereas post-fermentation is frequently used for the production of so-called plain type yogurt, etc., such as hard type (set type) yogurt without flesh of fruit and sweetener.

“Fermented milk” according to the present invention is a dairy product and a processed product obtained by fermenting milk, etc. with lactic acid bacteria or yeast, etc., and includes “fermented milk”, “dairy product fermented milk drink” and “fermented milk drink” and the like defined by the Ministerial Ordinance on Milk and Milk Products Concerning Compositional Standards, etc. The fermented milk produced according to the present invention may be, for example, yogurt. The fermented milk produced according to the present invention may be either pre-fermentation type yogurt wherein a raw material mix is fermented in a tank, etc., then filled in a container; or post-fermentation type yogurt wherein a raw material mix is filled in a container, then fermented. The fermented milk produced according to the present invention may be, for example, plain yogurt, set type yogurt (solid fermented milk), soft yogurt (pasty fermented milk), and drink yogurt (liquid fermented milk) and the like. In the production method of the present invention, since fermented milk having sufficient hardness can be produced, the method can be suitably utilized for set type yogurt.

The “raw material mix” according to the present invention includes milk, milk constituents, or a composition comprising milk constituents, which comprises fat. Milk constituents include, for example, raw milk, cow's milk, concentrated milk, whole milk powder, skimmed milk, concentrated skimmed milk, skimmed milk powder, partially-skimmed milk, concentrated partially-skimmed milk, partially-skimmed milk powder, sweetened condensed milk, sweetened condensed skimmed milk, evaporated milk, evaporated skimmed milk, whey, whey powder, desalted whey, desalted whey powder, whey protein concentrate (WPC), whey protein isolate (WPI), α-lactalbumin, 3-lactoglobulin, milk protein concentrate (MPC), casein, sodium caseinate, calcium caseinate, cream, fermented cream, compounded cream, cream powder, butter, fermented butter, buttermilk, buttermilk powder and butter oil, etc. The raw material mix may contain two or more of milk constituents. In addition to milk constituents, the raw material mix may further contain, for example, water, lipids, proteins, sugars, flavoring components, flavors, pigments, minerals (salts), vitamins and other additives for foods, etc. The raw material mix may also contain a gelatin liquid, etc. previously dissolved by heating.

In addition, the “raw material mix” according to the present invention comprises at least one type of an ultra-high temperature treated material, and examples of such material include milk-derived materials such as milk, a milk constituent, or a composition containing a milk constituent. Here, the ultra-high temperature treatment is not particularly limited as long as it is indicated as ultra-high temperature sterilization in the art, and it refers to, for example, heat sterilization at 115° C. to 150° C. for 1 to 10 seconds or at 120° C. to 150° C. for 1 to 5 seconds. The material sterilized by ultra-high temperature treatment according to the present invention is sterilized at least once in a thermal history of 115° C. or more, preferably 120° C. or more. The material that can be sterilized by ultra-high temperature treatment is not particularly limited as long as it is a milk-derived material that has been generally and conventionally sterilized at ultra-high temperature, and the examples thereof include, milk, skimmed milk, whole milk powder, partially-skimmed milk powder, skimmed milk powder, whole fat concentrated milk, concentrated partially-skimmed milk, concentrated skimmed milk, sweetened condensed milk, sweetened condensed skimmed milk, evaporated milk, evaporated skimmed milk, etc. Also in the present invention, the content of the material sterilized by ultra-high temperature treatment in a raw material mix is preferably 20% or more, and more preferably 50% or more.

Furthermore, the present invention enables to provide fermented milk having a curd strength which can withstand the transport without adding an additive for increasing the curd strength to the raw material mix. Additives for increasing the curd strength include stabilizers, gelling agents, thickeners, etc. as food additives. In the present invention, a raw material mix not containing additives for increasing the curd strength can be used.

In the present invention, the fat in a raw material mix can be homogenized by homogenizing the raw material mix containing the material sterilized by ultra-high temperature treatment. Although the method of homogenizing is not particularly limited, for example, a method of passing a raw material mix through a narrow gap by pressurizing and pressing, or a method of passing a raw material mix through a narrow gap by depressurizing and sucking, may be used. The average particle diameter of the fat in the homogenized raw material mix is decreased. The average particle diameter of the fat in the raw material mix can be adjusted by appropriately setting the pressure and flow rate of homogenization. The average particle diameter of the homogenized fat (fat ball) may also be evaluated by a laser diffraction type particle size distribution analyzer (for example, SALD-2200, Shimadzu Corporation).

In the present invention, the average particle diameter of the fat in a raw material mix is preferably 0.8 μm or less, more preferably 0.77 μm or less, further preferably 0.75 μm or less, further preferably 0.73 μm or less, further preferably 0.7 μm or less, further preferably 0.67 μm or less, and further preferably 0.65 μm or less. In the present invention, the average particle diameter of the fat in a raw material mix is preferably 0.2 μm or more, more preferably 0.25 μm or more, further preferably 0.3 μm or more, further preferably 0.35 μm or more, and further preferably 0.4 μm or more.

In addition, in the present invention, the standard deviation of the fat in a raw material mix is preferably 0.16 μm or less, more preferably 0.15 μm or less, further preferably 0.14 μm or less, and further preferably 0.13 μm or less. Also, the standard deviation of the particle diameter of a raw material mix of the present invention is preferably 0.01 μm or more, more preferably 0.05 μm or more, further preferably 0.08 μm or more, and further preferably 0.1 μm or more.

The pressure for homogenizing the raw material mix is required to be high so as to obtain a desired average particle diameter in the raw material mix, and is preferably 180 kg/cm² or more, more preferably 200 kg/cm² or more, further preferably 220 kg/cm² or more, further preferably 240 kg/cm² or more, further preferably 260 kg/cm² or more, further preferably 280 kg/cm² or more, and further preferably 300 kg/cm² or more. In addition, the pressure for homogenizing the raw material mix is preferably 800 kg/cm² or less, more preferably 700 kg/cm² or less, further preferably 600 kg/cm² or less, and further preferably 550 kg/cm² or less.

The pressure may be given in one stage or in multiple stages of two or more stages. For example, in the case of two stages, from the viewpoint of homogenization efficiency, it is preferable to relatively increase the pressure of the first stage and relatively lower the pressure of the second stage. Specifically, for example, it can be carried out in two stages of 80 to 790 kg/cm² and 10 to 100 kg/cm², and preferably carried out in two stages of 500 kg/cm² and 50 kg/cm².

The flow rate for homogenizing the raw material mix is not particularly limited as long as the intended homogenization is carried out; it is preferably 100 to 30000 kg/h, more preferably 150 to 25000 kg/h, further preferably 200 to 20000 kg/h, and further preferably 250 to 15000 kg/h.

Homogenization of the raw material mix may be performed between the period from the preparation of the raw material mix and before fermentation of the raw material mix at the latest, and if the fat in the raw material mix has a desired average particle diameter just before fermentation, homogenization may be carried out in one step or in two or more steps by combining other steps such as a heat sterilization step and a step for reducing dissolved oxygen. From the viewpoint of simplifying the manufacturing method, etc., homogenization of the raw material mix is preferably carried out in one step.

In the method of producing fermented milk of the present invention, in addition to homogenizing the raw material mix and fermenting the raw material mix, heat sterilization of the raw material mix and/or reduction of dissolved oxygen of the raw material mix may be appropriately carried out. Therefore, after preparation of the raw material mix and before fermentation, homogenization, heat sterilization, and reduction of dissolved oxygen may be carried out in an arbitrary order. For example, it can be performed in the order of preparation of raw material mix, homogenization, reduction of dissolved oxygen, heat sterilization, and fermentation; or in the order of preparation of raw material mix, heat sterilization, homogenization, reduction of dissolved oxygen, and fermentation. Preferably, reduction of dissolved oxygen concentration in the raw material mix is carried out prior to heat sterilization of the raw material mix, and particularly preferably, it is carried out in the order of preparation of raw material mix, homogenization of raw material mix, heat sterilization of raw material mix, reduction of dissolved oxygen concentration in raw material mix, and fermentation.

Heat sterilization method of raw material mix is not particularly limited. Low temperature/long time (LTLT) treatment, high temperature/short time (HTST) treatment, or ultra-high temperature (UHT) treatment can be used. Therefore, since the same sterilization conditions as milk, milk drink, etc. can be set, the same method and equipment as the products such as milk and milk drink can be used in the heat sterilization of raw material mix, production efficiency of various products in a dairy factory as a whole would not be lowered and installation of new equipment for each kind of products is not necessary.

The temperature at which the raw material mix is sterilized is not particularly limited as long as it is a sterilizable temperature, and is typically 63° C. to 150° C., preferably 115° C. to 150° C. By treating the raw material mix at a high temperature of 115° C. to 150° C., fermented milk with improved smoothness of mouth feel can be obtained. In addition, the time for sterilizing the raw material mix can be appropriately determined depending on the sterilization temperature, which is, for example, 1 to 1800 seconds. Regarding the combination of the temperature and time of heat sterilization, normalized or widely used heat sterilization conditions can be selected, and they are, for example, at 63° C. to 65° C. for 30 minutes (1800 seconds) (LTLT), at 72° C. to 75° C. for 15 seconds (HTST), at 115° C. to 150° C. for 1 to 10 seconds, or at 120° C. to 150° C. for 1 to 5 seconds (UHT), or the like.

Reduction of the dissolved oxygen concentration of the raw material mix is controlled such that the dissolved oxygen concentration of the raw material mix at the start of fermentation becomes lower than usual, for example, to be 5 ppm or less, preferably 4 ppm or less, more preferably 3 ppm or less, further preferably 2 ppm or less, and further preferably 1 ppm or less. By reducing the dissolved oxygen concentration of the raw material mix at the start of fermentation, the lactic acid acidity reaches a predetermined value quickly, so that the fermentation time can be shortened and production efficiency can be improved. In addition, compared to fermented milk without reducing the dissolved oxygen concentration, the texture of the fermented milk becomes dense and mellow.

A method of reducing the dissolved oxygen concentration of a raw material mix may be a method in which inert gas is injected into the raw material mix to replace the oxygen of the raw material mix with the inert gas, or a method in which the raw material mix is maintained in a low pressure or vacuum state to reduce the pressure and to deaerate, thereby removing the oxygen from the raw material mix. Here, as the inert gas, for example N₂ may be used, or a rare gas such as helium, neon, argon, or xenon may be used. In this case, the method of reducing the dissolved oxygen concentration of a raw material mix may be a method in which the holding time at the heating temperature is set to a predetermined value (somewhat longer time) in the method of heating (sterilizing) the raw material mix; for example, when the heating temperature of the raw material mix is 115° C. to 150° C., the holding time is preferably set to be 5 to 60 seconds, more preferably 5 to 30 seconds, and further preferably 5 to 10 seconds. At this time, the step of reducing the dissolved oxygen concentration of the raw material mix can be carried out simultaneously with the step of sterilizing the raw material mix.

Fermentation of raw material mix is carried out by adding a starter (inoculation). For fermentation, any starters such as lactic acid bacteria, bifidobacteria and yeast can be used. As a starter, one selected from lactic acid bacteria and yeast that are generally used in the production of fermented milk can be used alone, or two or more can be used in combination; and examples thereof are: Lactobacillus bulgaricus (Lactobacillus delbrueckii subsp. bulgaricus), Streptococcus thermophilus (Streptococcus salivarius subsp. thermophilus), Lactobacillus lactis, Lactobacillus gasseri, Lactobacillus plantarum, Lactobacillus casei, Lactobacillus acidophilus and Bifidobacterium, etc.

As a starter, from the viewpoints of being standardized as a yogurt starter in the Codex standard and the like, preferably it is a starter based on a mixed starter of Lactobacillus bulgaricus and Streptococcus thermophilus. Depending on the fermented milk to be actually obtained, Lactobacillus gasseri, Bifidobacteria, and yeast, etc. may be added to a yogurt starter used as a base.

The amount of a starter to be added may be an amount generally used in the production of fermented milk; for example, when a starter prepared by a subculture method (mother starter, bulk starter) is used, the amount of the starter relative to a raw material mix is, for example, 0.1 to 10 wt %, preferably 0.2 to 5 wt %, more preferably 0.5 to 4 wt %, further preferably 1 to 5 wt %, and particularly preferably 1 to 3 wt %. In a starter other than a starter prepared by a subculture method (mother starter, bulk starter) (for example, concentrated starter, freeze-concentrated starter, and freeze-dried starter, which are inoculated directly into raw material mix), its amount of addition may be appropriately adjusted depending on the number of bacteria in the starter. In addition, the method of adding the starter may be any method commonly used in the production of fermented milk; for example, it includes a method in which a starter is aseptically added to a raw material mix that is stored in a tank, etc., or a method in which a starter is added inline to a raw material mix that is flowing through the piping.

The method of fermenting a raw material mix may be a method in which a starter is added to a raw material mix, then the raw material mix is filled in containers and the containers are held in a fermentation chamber, or a method in which a starter is added to a raw material mix, then the raw material mix is filled in a tank, etc. and held within the tank, etc. And, in the method in which a raw material mix is filled in a tank, etc. and held within the tank, etc., after fermented milk is prepared in the tank, etc. and the fermented milk is stirred (the curd of the fermented milk is crushed), if necessary, the following method may be carried out to produce soft type yogurt: a method in which flesh of fruits, vegetables, preparations, sauce, sugar solutions, etc. are mixed and the resulting fermented milk is filled in containers. In addition, in the method in which a raw material mix is filled in a tank, etc. and held within the tank, etc., after fermented milk is prepared in the tank, etc. then the fermented milk is stirred (the curd of the fermented milk is crushed) and homogenized (the curd of the fermented milk is micronized), if necessary, the following method may be carried out to produce drink type yogurt: a method in which flesh of fruits, vegetables, preparations, sauce, sugar solutions, etc. are mixed and the resulting fermented milk is filled in containers.

The conditions for fermenting a raw material mix may be adjusted in consideration of the kind and the amount of addition of lactic acid bacteria to be added to the raw material mix, and the flavor, mouth feel, physical properties, etc. of the fermented milk to be actually obtained. The temperature and time at which the raw material mix is fermented can be appropriately set in accordance with purposes, etc. At this time, the temperature (fermentation temperature) at which the raw material mix is fermented is preferably 30° C. or more, more preferably 33° C. or more, further preferably 35° C. or more, and most preferably 37° C. or more. In addition, the temperature at which the raw material mix is fermented is preferably 50° C. or less, more preferably 45° C. or less, and further preferably 43° C. or less. By fermenting the raw material mix at 35° C. to 40° C., fermented milk with improved smoothness of mouth feel can be obtained, which is preferable. In addition, the time (fermentation time) for fermenting the raw material mix is preferably 1 to 24 hours, more preferably 1 to 12 hours, further preferably 2 to 8 hours, further preferably 2 to 6 hours, and further preferably 2 to 4 hours.

When fermenting the raw material mix, the lactic acid acidity (acidity) varies depending on the composition; when the milk solids-not-fat is approximately 8 wt %, preferably the lactic acid acidity reaches 0.5%, and more preferably reaches 0.6%. In addition, when the milk solids-not-fat is approximately 10 wt %, the lactic acid acidity (acidity) preferably reaches 0.6%, and more preferably reaches 0.7%. In addition, when the milk solids-not-fat is approximately 12 wt %, the lactic acid acidity (acidity) preferably reaches 0.7%, and more preferably reaches 0.8%. In addition, when the milk solids-not-fat is approximately 14 wt %, the lactic acid acidity (acidity) preferably reaches 0.8%, and more preferably reaches 1.0%. In addition, when the milk solids-not-fat is approximately 16 wt %, the lactic acid acidity (acidity) preferably reaches 1.0%, and more preferably reaches 1.1%. In addition, when the milk solids-not-fat is approximately 18 wt %, the lactic acid acidity (acidity) preferably reaches 1.1%, and more preferably reaches 1.3%. In addition, when the milk solids-not-fat is approximately 20 wt %, the lactic acid acidity (acidity) preferably reaches 1.2%, and more preferably reaches 1.4%.

In the present invention, fermentation of a raw material mix may be carried out in a container of product. For example, after homogenizing and sterilizing the raw material mix, a starter may be added to the raw material mix and then the raw material mix may be filled in a container. Here, the container may be any container commonly used in the production of fermented milk (dairy product), for example, it may be a container made of plastic, glass, and paper, etc.

The present invention can provide fermented milk having a hardness (strength, curd tension) that can withstand vibrations during distribution without being crushed by impact, etc. during transport, and is excellent in smoothness of mouth feel.

“Hardness” of the fermented milk of the present invention may be evaluated as a measurement value of the breaking point of yogurt knife in a curdmeter (for example, MAX ME-500, I. Techno Engineering); specifically, it may be evaluated by measuring the breaking point by the curdmeter with a measurement temperature of 5° C. to 10° C. and a load of 100 g.

In the fermented milk of the present invention, as long as hardness is 26 g or more, it is effective to suppress crushing due to impact etc. during transport while maintaining the texture stably during distribution. The hardness of the fermented milk of the present invention is preferably 30 g or more, and more preferably 32 g or more. The hardness of the fermented milk of the present invention is preferably 100 g or less, more preferably 90 g or less, further preferably 80 g or less, and further preferably 70 g or less.

“Excellent in smoothness of mouth feel” means that the curd has a dense texture and gives no grainy feeling on the tongue when placed in the oral cavity. “Smoothness of mouth feel” in fermented milk may be evaluated as a measured value of the penetration angle of yogurt knife in a curdmeter (for example, MAX ME-500, I. Techno Engineering); specifically, it may be evaluated by the following measurement: setting the measurement temperature at 5° C. to 10° C. and the load at 100 g, the angle between the tangent directed to the breaking point passing through the origin and the time-load curve after the breaking point is measured. When this angle is large, the fermented milk is evaluated as fermented milk having grainy rough mouth feel, and when this angle is small, it is evaluated as fermented milk having smooth mouth feel.

In the fermented milk of the present invention, the penetration angle of yogurt knife may be less than 60 degrees, and smooth feeling on the tongue is effectively improved while the texture is stably maintained during distribution. In other words, the penetration angle of yogurt knife of the fermented milk of the present invention is preferably less than 60 degrees, more preferably less than 56 degrees, further preferably less than 52 degrees, and further preferably less than 48 degrees. In addition, the penetration angle of yogurt knife of the fermented milk of the present invention is preferably 10 degrees or more, more preferably 15 degrees or more, further preferably 20 degrees or more, and further preferably 25 degrees or more.

“Smoothness of mouth feel” in fermented milk may be evaluated as a measured value of the average particle diameter of fermented milk (final products, intermediate products) after stirring, and the average particle diameter of fermented milk after stirring is measured and evaluated by a laser diffraction type particle size distribution analyzer (for example, SALD-2200, Shimadzu Corporation). When the particle diameter after stirring is large, fermented milk is evaluated as the one having grainy rough mouth feel. When the particle diameter after stirring is small, fermented milk is evaluated as the one having smooth mouth feel.

In the fermented milk of the present invention, the average particle diameter after stirring may be 43 μm or less, and smooth feeling on the tongue is effectively improved while the texture is stably maintained during distribution. In other words, the average particle diameter of the fermented milk of the present invention after stirring is preferably 43 μm or less, more preferably 42 μm or less, further preferably 41 μm or less, and most preferably 40 μm or less.

The fermented milk of the present invention may be yogurt, which may be any of pre-fermentation type yogurt and post-fermentation type yogurt, and it is preferably post-fermentation type yogurt. The fermented milk of the present invention may be a plain type yogurt, which may be any of set type yogurt (solid fermented milk), soft type yogurt (pasty fermented milk), and drink type yogurt (liquid fermented milk), and it is preferably plain type yogurt, which is a set type yogurt.

The fermented milk of the present invention may be packed in a container. “Packed in a container” means that it has been filled and sealed in a container. The container may be any container commonly used in the production of fermented milk (dairy products), for example, it may be a container made of plastic, glass, paper, etc. The fermented milk of the present invention may be the one wherein a raw material mix (which also includes a fermented milk base material, that is in the state that a lactic acid bacteria starter, etc. is added in the raw material mix) is filled in a container, then the raw material mix is fermented in the container (post-fermentation type), or the one wherein a raw material mix in a tank, etc. is fermented before it is filled in a container (after fermenting the raw material mix in the tank, etc., the fermented milk is filled in a container) (pre-fermentation type).

In the present invention, the milk solids-not-fat of the raw material mix and/or fermented milk (final products and/or intermediate products) is preferably 8 wt % or more, more preferably 8.5 wt % or more, further preferably 9 wt % or more, and more preferably 9.5 wt % or more. In addition, the milk solids-not-fat of the raw material mix and/or fermented milk is preferably 30 wt % or less, more preferably 25 wt % or less, further preferably 20 wt % or less, and further preferably 18 wt % or less. In the present invention, the fat (lipid) of the raw material mix and/or fermented milk is preferably 0.5 wt % or more, more preferably 1 wt % or more, further preferably 1.5 wt % or more, further preferably 2 wt % or more, further preferably 2.5 wt % or more, further preferably 3 wt % or more, and further preferably 3.1 wt % or more. In addition, the fat (lipid) of the raw material mix and/or fermented milk is preferably 8 wt % or less, more preferably 7 wt % or less, further preferably 6 wt % or less, further preferably 5 wt % or less, further preferably 4.5 wt % or less, further preferably 4 wt % or less, and further preferably 3.5 wt % or less.

### EXAMPLES

Embodiments of the present invention will be described in more detail while showing examples below; however, the present invention is not limited to the following examples.

### [Measurement Method of Average Particle Diameter]

The average particle diameter and standard deviation of a raw material mix were measured using a laser diffraction type particle size distribution analyzer SALD-2200 (Shimadzu Corporation). Specifically, the raw material mix was diluted with ion exchanged water to adjust the maximum value of the distribution of light intensity of diffraction/scattering to be 35 to 75% (absolute value: 700 to 1500). Using software WingSALD II for the particle size distribution analyzer, distribution of the light intensity was analyzed and average fat particle diameter and standard deviation were determined.

### [Measurement Method of Hardness of Fermented Milk]

Hardness (strength or curd tension) of fermented milk was measured using a curdmeter MAX ME-500 (I. Techno Engineering). Specifically, a yogurt knife with 100-g weight attached was placed on the top surface of the fermented milk, the fermented milk was continuously elevated while applying a load at about 2 g/sec, and with the elapsed time of this load application, measured values of this load are represented by a curve. Here, elapsed time (second) of this load application is represented on the vertical axis, measured value of this load is represented on the horizontal axis, and 10 g on the vertical axis and 4 seconds on the horizontal axis are expressed as the same distance. Then, when the fermented milk breaks, the yogurt knife invades from the top surface of the fermented milk, generating an inflection point (breaking point) in this time-load curve, and the value of the load to reach this breaking is defined as an indicator of the hardness (g).

### Production Example 1

A cream sterilized at 125° C., skimmed milk powder prepared from skimmed milk sterilized by UHT treatment at 125° C. for 15 seconds, and water were mixed to prepare a raw material mix (yogurt mix) so as to achieve the fat content of 3.0 wt % and the milk solids-not-fat of 9.5 wt %. The prepared raw material mix was heated to about 80° C. and then homogenized in two stages of 500 kg/cm² and 50 kg/cm². Subsequently, the homogenized raw material mix was heat sterilized at 95° C. (batchwise sterilization by heating at a reaching temperature of 95° C.) and then cooled to about 10° C. The obtained raw material mix had an average fat particle diameter of 0.58 μm.

After warming the obtained raw material mix to 43° C., nitrogen (N₂) was injected to reduce the dissolved oxygen concentration (DO) of the raw material mix to 5 ppm, and then a lactic acid bacteria starter (Lactobacillus bulgaricus and Streptococcus thermophilus isolated from Meiji Bulgaria Yogurt LB81) was added (inoculated) at 3 wt %. Thereafter, the mixture was filled in a plastic cup container (100 g capacity) and a paper cup container (450 g capacity), allowed to stand for about 3 hours in a fermentation chamber (43° C.) until the lactic acid acidity reached 0.70%, and then cooled in a refrigerating room (10° C. or lower) to produce set type yogurt (Example 1).

### Production Example 2

A cream sterilized at 125° C., skimmed milk powder prepared from skimmed milk sterilized by UHT treatment at 125° C. for 15 seconds, and water were mixed to prepare a raw material mix (yogurt mix) so as to achieve the fat content of 3.0 wt % and the milk solids-not-fat of 9.5 wt %. The prepared raw material mix was heated to about 80° C. and then homogenized in two stages of 100 kg/cm² and 50 kg/cm². Subsequently, the homogenized raw material mix was heated (sterilized) at 130° C. for 2 seconds, and then cooled to about 10° C. The obtained raw material mix had an average fat particle diameter of 0.94 μm.

After warming the obtained raw material mix to 43° C., nitrogen (N₂) was injected to reduce the dissolved oxygen concentration (DO) of the raw material mix to 5 ppm, and then a lactic acid bacteria starter (Lactobacillus bulgaricus and Streptococcus thermophilus isolated from Meiji Bulgaria Yogurt LB81) was added (inoculated) at 3 wt %. Thereafter, the mixture was filled in a plastic cup container (100 g capacity), allowed to stand for about 3 hours in a fermentation chamber (43° C.) until the lactic acid acidity reached 0.70%, and then cooled in a refrigerating room (10° C. or lower) to produce set type yogurt (Comparative example 1).

Curd strength of the obtained set type yogurt of Example 1 and Comparative example 1 was measured. Curd strength was measured using Neo-curdmeter M302 (I. Techno Engineering).

The curd strength of Example 1 reached 39 g, which was larger than the curd strength (24 g) of Comparative example 1. From this result, it was confirmed that the yogurt of Example 1 satisfied the target hardness sufficiently and had hardness capable of resisting the impact at the time of distribution of the product.

Namely, the yogurt of the present invention is considered to be able to achieve the desired hardness and smoothness of mouth feel, by reducing the average particle diameter of the fat in the raw material mix.

### Production Example 3

Raw milk was heat sterilized (pasteurized) at 65° C. for 30 minutes and then homogenized in two stages of 350 kg/cm² and 50 kg/cm². It was then cooled to about 10° C. The average fat particle diameter of the obtained raw material mix was 0.74 μm.

After warming the obtained raw material mix to 43° C., nitrogen (N₂) was injected to reduce the dissolved oxygen concentration (DO) of the raw material mix to 5 ppm, and then a lactic acid bacteria starter (Lactobacillus bulgaricus and Streptococcus thermophilus isolated from Meiji Bulgaria Yogurt LB81) was added (inoculated) at 3 wt %. Thereafter, the mixture was filled in a plastic cup container (100 g capacity) and a paper cup container (450 g capacity), allowed to stand for about 3 hours in a fermentation chamber (43° C.) until the lactic acid acidity reached 0.70%, and then cooled in a refrigerating room (10° C. or lower) to produce set type yogurt (Reference example 1).

### Production Example 4

Fresh milk, skimmed milk powder prepared from skimmed milk sterilized by UHT treatment at 125° C. for 15 seconds, and water were mixed to prepare a raw material mix (yogurt mix) so as to achieve the fat content of 3.0 wt % and the milk solids-not-fat of 9.5 wt %. The prepared raw material mix was heated to about 80° C. and then homogenized in two stages of 100 kg/cm² and 50 kg/cm². Subsequently, the homogenized raw material mix was heat sterilized at 95° C. (batchwise sterilization by heating at a reaching temperature of 95° C.), and then cooled to about 10° C. The obtained raw material mix had an average fat particle diameter of 1.21 μm.

After warming the obtained raw material mix to 43° C., nitrogen (N₂) was injected to reduce the dissolved oxygen concentration (DO) of the raw material mix to 5 ppm, and then a lactic acid bacteria starter (Lactobacillus bulgaricus and Streptococcus thermophilus isolated from Meiji Bulgaria Yogurt LB81) was added (inoculated) at 3 wt %. Thereafter, the mixture was filled in a plastic cup container (100 g capacity), allowed to stand for about 3 hours in a fermentation chamber (43° C.) until the lactic acid acidity reached 0.70%, and then cooled in a refrigerating room (10° C. or lower) to produce set type yogurt (Reference comparative example 1).

### Test Example 1

Curd strength of the obtained set type yogurt of Reference example 1 and Reference comparative example 1 was measured. Curd strength was measured using Neo-curdmeter M302 (I. Techno Engineering).

The curd strength of Reference example 1 reached 55 g and it was larger than the curd strength (24 g) of Reference comparative example 1. From this result, it was confirmed that the yogurt of Reference example 1 satisfied the target hardness sufficiently and had hardness capable of resisting the impact at the time of distribution of the product.

### Test Example 2

A raw material mix was prepared in the same manner as Reference example 1, fermented after reducing the dissolved oxygen concentration (DO) to 5 ppm, and the fermentation time until lactic acid acidity reached 0.70% was measured (Reference example 2).

In addition, a raw material mix was prepared in the same manner as Reference example 1, fermented without reducing the dissolved oxygen concentration (DO), and the fermentation time until lactic acid acidity reached 0.70% was measured (Reference example 3).

The results are shown in Table 1.

TABLE 1
Effect of deoxygenated fermentation on reduction of
fermentation time
Time to reach lactic acid acidity of 0.7%

| Reference example 2 | Reference example 3 |
|---|---|
| 3 hours | 4 hours |

It was confirmed that the fermentation time can be shortened by conducting deoxygenation treatment before fermentation. The shorter fermentation time means that the time required for production is shorter and the production efficiency is higher.

### INDUSTRIAL APPLICABILITY

The present invention can be used for the production of fermented milk such as yogurt, and in particular it can be suitably used for the production of set type yogurt. In addition, the present invention broadens the application of materials sterilized by ultra-high temperature treatment which are generally difficult to utilize from the viewpoint of coagulability of yogurt, as materials for fermented milk such as yogurt, etc.

1. A method for producing fermented milk using a material sterilized by ultra-high temperature treatment, comprising homogenizing the fat of a raw material mix containing the material sterilized by ultra-high temperature treatment, and conducting fermentation.

2. The method according to claim 1, wherein the material sterilized by ultra-high temperature treatment is one or more selected from the group consisting of milk, concentrated milk, whole milk powder, skimmed milk, concentrated skimmed milk, skimmed milk powder, partially-skimmed milk, concentrated partially-skimmed milk, partially-skimmed milk powder, cream and butter.

3. The method according to claim 1 or 2, wherein the temperature of the ultra-high temperature treatment is 120° C. to 150° C.

4. The method according any one of claims 1 to 3, wherein the fat in the raw material mix is homogenized such that its average particle diameter becomes 0.8 μm or less.

5. The method according to any one of claims 1 to 4, further comprising heat sterilizing the raw material mix.

6. The method according to any one of claims 1 to 5, further comprising reducing the dissolved oxygen concentration of the raw material mix.

7. The method according to claim 6, comprising reducing the dissolved oxygen concentration of the raw material mix before heat sterilizing the raw material mix.

8. The method according to claim 6 or 7, comprising reducing the dissolved oxygen concentration of the raw material mix before fermenting the raw material mix.

9. The method according to any one of claims 1 to 8, wherein the fermentation of the raw material mix is carried out in a container of the product.

10. Fermented milk produced by the method according to any one of claims 1 to 9.

11. The fermented milk according to claim 10, wherein the fermented milk is a set type yogurt.

12. The method according to claim 11, wherein the hardness is 26 g or more.
